(19) United States
(12) Patent Application Publication
(10) Pub. No.: US 2009/0069425 A1
(43) Pub. Date: Mar. 12, 2009

(54) Active composition for treatment or prevention of problems related to a disturbance of the biological clock

(75) Inventor: Jean-Christophe Anton, Strasbourg (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Persee Medica, a corporation of France, Bailly (FR)

(21) Appl. No.: 11/920,013

(22) PCT Filed: May 12, 2006
(86) PCT No.: PCT/FR2006/001079

§ 371 Date: Jan. 10, 2008

(30) Foreign Application Priority Data
May 13, 2005 (FR) 0551254

(51) Int. Cl. A61K 31/201

(52) U.S. Cl. 514/560

(57) ABSTRACT

A composition contains polyunsaturated fatty acids and flavonoids as active substances. A method treats disorders caused by disturbance of a biological clock in humans and animals and includes administering a therapeutically effective amount of the composition to a human or animal. A method regulates the biological clock in a mammal and includes administering a therapeutically effective amount of the composition to the mammal.

### RELATED APPLICATION

This is a §371 of International Application No. PCT/FR2006/001079, with an international filing date of May 12, 2006 (WO 2006/120360 A2, published Nov. 16, 2006), which is based on French Patent Application No. 05/51254, filed May 13, 2005.

### TECHNICAL FIELD

This disclosure relates to the treatment and prevention of problems caused by disturbance of the biological clock in humans and, when appropriate, in animals. More specifically, the disclosure concerns subjects whose waking—sleep cycle is disturbed, with as the main signs difficulty in falling asleep, poor sleep quality, difficulty in waking up, major fatigue during the daytime time and, consequently, memory disturbances, difficulties in concentration and in orientation in space.

### BACKGROUND

Biological rhythms have been observed in humans and in the animal and plant kingdom at all organizational levels: ecosystem, population, individuals, organ systems, isolated organs, tissues, cells and subcellular fractions. Chronobiology has emerged from this research, the aim of which is study and quantification of the mechanisms of the biological temporal structure. The biological clock is closely related to the waking—awakening rhythms and consequently to sleep.

The body's dominant biological clock is located in the suprachiasmatic nuclei. It is responsible for forming and distributing oscillations to pacemakers. The lack of scientific research leads one to devote interest (by exclusion) to the dominant biological clock which controls the circadian rhythm.

The oscillations produced by the biological clock give rise to the production of hormones. The most well known and studied to date is melatonin. The “hormonal” nature of the biological clock results in major variabilities between individuals. Indeed, many factors such as age, sex, weight, height and ethnic origin influence hormonal status. Other external factors (environment) also play a role. Under physiological conditions, the biological clock may be desynchronized by external factors (time difference, night work) and by internal factors (aging, illness, depression, surgical operations . . . ). Within this context, melatonin plays a role as a synchronizer of the biological clock.

Other studies confirm the importance of melatonin. A recent study shows that aging results in a reduction in melatonin synthesis. This reduction would seem to be counterbalanced however by compensatory regulation mechanisms (increase in the expression of the melatoninergic receptors). The action of melatonin on aging therefore still remains a controversial subject and scientific proof is lacking. On the other hand, desynchronization of the biological clock by reduction in melatonin concentrations in certain patients is a studied and validated phenomenon.

Melatonin is synthesized from serotonin. It is mainly broken down in the liver under the influence of enzymes controlled by the cytochrome CYP1A2. Three melatonin receptors have been cloned to date, but unlike other families of receptors, it is still not possible to link a physiological function or a behavior to a specific receptor. The melatonin synthesis represented in FIG. 1 follows a main physiological pathway under the influence of two hormones (AA-NAT and HIOMT). Several lines of approach are possible to compensate for melatonin deficiency: supply or promotion of production of precursors of melatonin, direct supply of melatonin or promotion of the latter's production and reduction in degradation of melatonin.

The biological clock closely controls the rhythms and alternation of waking—awakening. Under certain circumstances, the biological clock may be desynchronized by external factors (time difference, night work) and by internal factors (aging, illness, depression, surgical operations . . . ). The initial manifestation of this desynchronization affects sleep and is more specifically indicated by poor sleep quality, difficulties in falling asleep and decreased daytime vigilance.

### SUMMARY

I provide a composition containing polyunsaturated fatty acids and flavonoids as active substances.

I also provide a method of treating disorders caused by disturbance of a biological clock in humans and animals including administering a therapeutically effective amount of the composition to a human or animal.

I further provide a method of regulating the biological clock in a mammal including administering a therapeutically effective amount of the composition to the mammal.

### BRIEF DESCRIPTION OF THE DRAWINGS

Reference will be made below to the appended drawings in which:

FIG. 1 represents a melatonin synthesis pathway;

FIG. 2 is a graph of the evolution in the criteria (D0 versus D30); and

FIG. 3 is a graph of the evolution in the criteria (D0 versus D30 versus D60, TOTAL item on the lefthand axis, other items on the righthand axis).

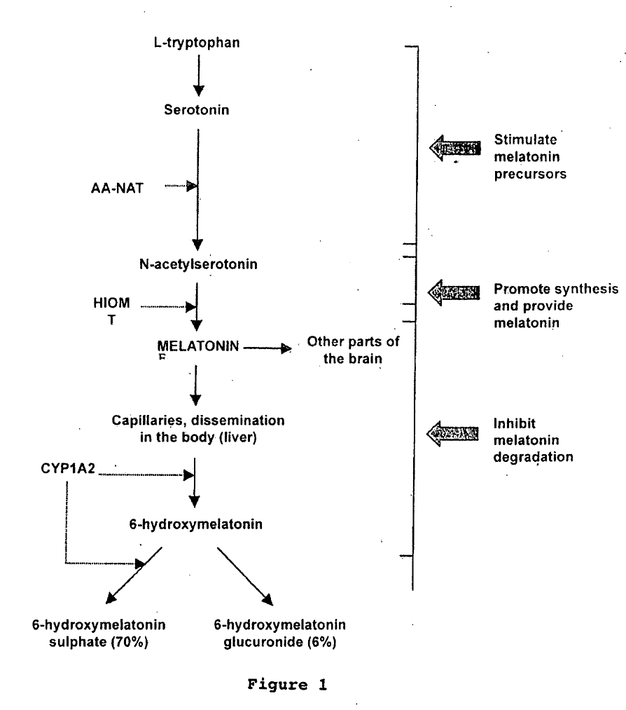

### DETAILED DESCRIPTION

I provide compositions capable of effectively counteracting the disorders caused by disturbance of the biological clock. More specifically, my compositions comprise an original combination of natural active substances that promote melatonin synthesis from the latter's precursors and increase its bioavailability by acting on its metabolism. The combined action of these various active substances encourages rebalancing of the biological clock. Furthermore, certain active substances contained in the composition also play a direct role in sleep due to their sedative properties.

I thus provide nutritional supplements or pharmaceutical compositions comprising polyunsaturated fatty acids and flavonoids as active substances.

This combination is remarkable in that it has a physiological action at the various stages of the metabolic pathway of melatonin synthesis and degradation. It encourages melatonin synthesis from its precursors and increases its bioavailability by acting on its metabolism.

The polyunsaturated fatty acids in the composition are preferably alpha linolenic acid (C18:3n-3) and linoleic acid (C18:2n-6).

Advantageously, the alpha-linolenic and linoleic acids in the composition are present in the form of one or several vegetable oils, such as hemp or soybean oil.

According to one aspect, the composition involves a weight ratio of alpha linolenic acid (C18:3n-3)/linoleic acid (C18:2n-6) ranging from 0.01 to 10, particularly from 0.1 to 1 and specifically from 0.2 to 0.8, or indeed 0.25 to 0.5.

The alpha-linolenic and linoleic acids increase serotonin synthesis in the brain and improve its bioavailability by inducing its release in the platelets. In parallel, linoleic acid, by increasing the cyclic AMP concentrations, stimulates the two enzymes AA-NAT (responsible for conversion of serotonin into N-acetyl serotonin) and HIOMT (responsible for conversion of N-acetyl serotonin into melatonin). Consequently, polyunsaturated fatty acids could encourage an increase in the melatonin concentrations by influencing the availability of the precursor (serotonin) and its metabolization into melatonin (stimulation of the enzymes responsible for conversion).

The flavonoids in the composition are those contained in an extract of Humulus lupulus ps). They have inhibiting properties on the cytochrome CYP1A2, responsible for metabolization melatonin. Inhibition of the enzyme that degrades melatonin into its inactive metabolite tends to reinforce the activity of the hormone by simply increasing its concentration and its bioavailability for its receptors. Other substances present in the extract such as humulone and lupulone also have sedative properties in humans.

Specifically, the composition comprises a weight ratio of linoleic acid and linolenic acid/dry extract of hop cones ranging from 1 to 20, particularly 5 to 15 and specifically from 8 to 12.

The compositions are prepared by incorporating a dry extract of hop cones (female inflorescences of Humulus lupulus) in vegetable oils rich in polyunsaturated fatty acids. Consequently, the quantity of extract and oil in the composition is such that allows a daily supply of:

Humulus lupulus (dry extract of hop cone powder): 20 to 1000 mg and preferably on the order of 100 mg;

vegetable oil(s) rich in polyunsaturated fatty acids (hemp and soybean oil): 400 to 3000 mg and preferably on the order of 866 mg, corresponding to:
approx. 300 to 2600 mg and preferably on the order of 540 mg of linoleic acid,
approximately 60 to 600 mg and preferably on the order of 160 mg of alpha-linolenic acid.

This daily intake may be provided by two administrations of the compositions.

As a preferred example, one composition comprises from:
10 to 500 mg and preferably on the order of 50 mg of extract of Humulus lupulus, and
200 to 1500 mg and preferably 433 mg of vegetable oil(s) rich in polyunsaturated fatty acids (hemp and soybean oil), corresponding to:
   approx. 150 to 1300 mg and preferably on the order of 370 mg of linoleic acid,
   approx. 30 to 300 mg and preferably on the order of 80 mg of alpha-linolenic acid.

The compositions may furthermore comprise one or several other substances capable of supplementing or increasing the action of the above active substances. One may mention for example:
piperine and piperidine, or furthermore plant extracts containing them,
active substances known in disturbances of the biological clock such as melatonin, N-acetyl serotonin, serotonin and tryptophan,
active substances allowing stimulation of arylalkylamine-N-acetyltransferase (AA-NAT) and hydroxyindole-O-methyltransferase (HIOMT) to yield melatonin,
sedative active substances, such as valerian for example,
antidepressant and hypnotic, calming or relaxing active substances,
vitamins and minerals,
tonic active substances to combat daytime fatigue, such as ginseng for example,
active substances to reinforce the immune system weakened by disturbances in the biological clock . . . ,
excipients adapted to the various different pharmaceutical forms in which the compositions may be presented and promoting for example the bioavailability of the active substances or increasing their efficacy.

The compositions may be presented in any type of solid form (tablets, capsules, soft capsules, cachets, sachets, powders, granulates, etc.) and liquid form (solutions, syrups, drops, emulsions, etc.).

As indicated above, the compositions are intended for treatment and/or prevention of disorders caused by disturbance of the biological clock in humans and, when appropriate, animals. The combination of polyunsaturated fatty acids and flavonoids described above is therefore very specifically useful for preparation of a drug designed to synchronize the biological clock in subjects having poor quality sleep, for example.

The compositions are indicated in subjects whose waking—sleep cycle is disturbed, with as the main signs sleep disturbances, difficulty in falling asleep, poor sleep quality, difficulty in waking up, major fatigue during the daytime and consequently memory disturbances, difficulties in concentration and orientation in space.

Among sleep disturbances, one may mention:
dyssomnia: insomnia of psychological origin (inability to sleep at night), altitude insomnia, insomnia of external origin (extrinsic), sleep disturbances related to alcohol or drugs and narcolepsy,
parasomnia: sleep disturbances accompanied by nocturnal awakening, but without any significant disturbance in sleep or impairment of daytime vigilance. Parasomnia includes sleepwalking, night terrors, sleep disturbances associated with REM sleep and nocturnal bruxism, and
sleep disturbances of psychiatric or neurological origin or related to other illnesses.

I also use a combination of polyunsaturated fatty acids and flavonoids as an agent to combat disorders caused by disturbance of the biological clock in compositions, particularly in a nutritional supplement or pharmaceutical composition or for preparation of compositions intended to combat the disorders caused by disturbance of the biological clock.

I furthermore use a combination of polyunsaturated fatty acids and flavonoids as an agent for regulation of the biological clock in compositions or for preparation of compositions intended to regulate the biological clock.

Other advantages and characteristics of my methods and compositions will be apparent from the following examples concerning preparation of a composition and a study conducted in 33 patients.

This study shows that a composition, denoted HB1 hereafter, is effective against sleep disturbances with a highly significant improvement in the total score on the SIRS scale (Sleep Impairment Rating Scale). The subjects participating in the study had their sleep markedly improved for all the different items measured. This improvement is greater than 40% after one month of administration of HB1 for the overall score, with a major improvement in sleep quality (43%), satisfaction with sleep (44%) and a reduction in the interference of sleep problems with the patients' everyday life (37%). The results are consolidated and increased after an additional month of administration of the product.

“On the order of X” or “approximately X” means a range of values extending from X less 15% to X plus 15%, particular from X less 10% to X plus 10%, specifically from X less 5% to X plus 5% and indeed from X less 2% to X plus 2%.

### EXAMPLE 1

### Preparation of a Composition (HB1)

A composition is prepared by incorporating a dry extract of hop cones (female inflorescences of Humulus lupulus) in vegetable oils rich in polyunsaturated fatty acids. Consequently, the quantity of extract and oil in the composition is such that allows a daily supply of:
Humulus lupulus (dry extract of hop cone powder): 100 mg;
vegetable oils of hemp and soybeans (rich in polyunsaturated fatty acids): 866 mg.

### EXAMPLE 2

### Study of the Properties of HB1 On Sleep

### 1) Analysis of Insomnia.

The main criterion of this study was to measure sleep improvement in subjects with difficulties in falling asleep, poor sleep quality and consequently reduced daytime vigilance. The efficacy of HB1 was assessed by using a questionnaire allowing qualification and quantification of insomnia according to the SIRS scale (Sleep Impairment Rating Scale).

Insomnia is intended to mean a reduction in the quality and duration of sleep, or recovery. Insomnia is a very frequent complaint that results in major social and economic costs. The prevalence of insomnia is established as 27% on an occasional basis and 9% on a chronic level. Insomnia is accompanied by many symptoms that interfere with an individual's daily functioning.

Insomnia may be assessed by subjective methods (clinical interview, questionnaire, sleep diary) and objective methods (polysonographic recording, actigraphy). The clinical interview makes it possible to obtain information about the nature, history and severity of the sleep difficulties and about the possible causes of these difficulties. Even though essential, this type of assessment demands a great deal of time on the clinician's part and a certain amount of experience to conduct the interview properly. Keeping a sleep diary or a logbook completed by the patient every day makes it possible to collect useful information about the sleep habits. Administration of questionnaires may also facilitate the process of assessing insomnia while minimizing the factors of time and cost. The latter make it possible to identify and screen for sleep disturbances, the quantity and severity and the impact of the insomnia. Questionnaires provide rapid and general information about the patient's subjective perception concerning his/her sleep difficulties.

In this respect, the Sleep Impairment Rating Scale (SIRS) makes it possible to obtain an overall index of the deterioration or improvement in sleep. The patient assesses the severity of his/her difficulties in falling asleep, his/her nocturnal awakenings and premature awakenings, the degree of satisfaction with his/her sleep and the degree of interference with his/her daily functioning, the appearance of the deterioration related to his/her sleep problem and the level of concern that these difficulties cause him/her. The clinician and a significant person from the patient's entourage can complete these questionnaires to obtain additional indices that more precisely measure the severity of the problem. This questionnaire is very useful in clinical medicine and research since it makes it possible to rapidly obtain a subjective index of the patient's distress with regard to his/her sleep disturbances. Furthermore, it proves to be an instrument that is sensitive to the changes following treatment of insomnia. The SIRS scale questionnaire is given in Tables 1 to 5 below:

TABLE 1
please score the severity of your insomnia problems
(for the past two weeks) from 0 to 4.

| | None | Slight | Moderate | Severe | Very severe |
|---|---|---|---|---|---|
| a) Difficulty in | 0 | 1 | 2 | 3 | 4 |
| falling asleep | | | | | |
| b) Difficulty in | 0 | 1 | 2 | 3 | 4 |
| staying asleep | | | | | |
| c) Problem of waking | 0 | 1 | 2 | 3 | 4 |
| too early | | | | | |

TABLE 2
what is your level of satisfaction with your sleep?

| | | | | Highly |
|---|---|---|---|---|
| Highly satisfied | Satisfied | Indifferent | Dissatisfied | dissatisfied |
| 0 | 1 | 2 | 3 | 4 |

TABLE 3
to what extent do you consider that your sleep problems interfere
with your daily life (tiredness during the day, working capacity,
management of daily tasks, concentration, memory, mood)?

| Not at all | Slightly | Moderately | Markedly | Tremendously |
|---|---|---|---|---|
| 0 | 1 | 2 | 3 | 4 |

TABLE 4
to what extent do you think that your sleep problems
are noticeable to others in your quality of life?

| Not at all | Slightly | Moderately | Markedly | Tremendously |
|---|---|---|---|---|
| 0 | 1 | 2 | 3 | 4 |

TABLE 5
to what extent does your sleep problem
cause you anxiety and depression?

| Not at all | Slightly | Moderately | Markedly | Tremendously |
|---|---|---|---|---|
| 0 | 1 | 2 | 3 | 4 |

Assessment is performed by a system of addition of the scores of the different questions according to the formula:

Total score=score of question 1a+1b+1e+2+3+4+5

It is considered that a total score of 0 to 7 indicates a state of total absence of insomnia, of 8 to 14 problems of latent insomnia, of 15 to 21 moderate insomnia and from 22 to 28 severe insomnia.

### 2) Procedure

2.1) Inclusion visit (D0)

At the inclusion visit, the assessment physician proposes the study to patients forming part of his/her clientele. The inclusion criteria must be complied with, i.e., healthy male or female volunteers, of 16 to 50 years of age, whose biological clock is desynchronized (the waking—sleep cycle is disturbed), with as the main signs difficulty in falling asleep, poor sleep quality, difficulty in waking up owing to falling asleep late or on the other hand waking up too early, decreased vigilance during the daytime characterized by major fatigue, memory disturbances, difficulties in concentration and orientation in space.

In proposing the study, the assessment physician must ensure that the subject is faced with a sleep disturbance in his/her professional or family life indicated by a situation of insomnia and that the subject is prepared to accept the study.

The patient completes the scale by scoring the different questions according to intensity. The assessment physician must calculate the total score corresponding to the sum of the answers to the questions to validate inclusion. If the score is ≧to 15, he/her continues with inclusion of the subject.

The subject receives two capsules every evening, approximately two hours before retiring to bed, for a 30-day period.

### 2.2) Intermediate Visit

At the second appointment, the patient fills in the second part of the results book. The patient takes two capsules every evening, approximately two hours before retiring to bed, for a further 30-day period.

### 2.3) End of Study Visit

At the third appointment, the patient fills in the third part of the results book. The experimenting physician collects all the results books for analysis.

### 3) Results

### 3.1) Inclusion Visit Analysis (D0)

42 patients were included in the study. The subjects' average age was 48 years and the majority of the subjects participating in the study were women (more than 80%). The inclusion criteria were fulfilled, i.e., male and female healthy volunteers, of 16 to 50 years of age, experiencing sleep disturbances and reduced vigilance during the daytime.

The questionnaire was constructed in five parts and was to be completed by the physician during questioning of the subject. The first item related to the problems of insomnia and particularly difficulty in falling asleep, difficulty in staying asleep and problems of waking too early. All these three questions were grouped together in a subtotal designated “sleep quality.” The second item concerned the level of satisfaction with sleep, the third interference of the sleep problems during the daytime (tiredness during the day, working capacity, management of daily tasks, concentration, memory and mood), the fourth influence of the sleep problems on quality of life and finally the fifth the anxious and depressive character of the sleep problems. The questionnaire complied with the study protocol.

Among the 42 patients included at D0, 9 patients did not attend the intermediate visit at D30 and the end of study visit at D60. These patients were excluded from the study and it was possible to perform the analysis of the results on a total of 33 patients. There were no apparent reasons for these patients' withdrawal from the study. Some patients contacted by telephone mentioned that the product was active and that they did not see any need to consult again.

On inclusion, the main results (Table 1) indicate that the mean of the sum of all the items (19) is greater than the set limit (≧15) necessary to validate inclusion. Depending on the items, there are significant disparities between the patients with relatively high variance scores. However, when one measures the sum of several items such as the items “difficulty in falling asleep, staying asleep, problem of waking too early” (corresponding to the item “Sleep quality”), measurement of the variance is lower and indicates better uniformity of the patients (Table 2).

TABLE 1

| | Total | Mean | Variance |
|---|---|---|---|
| Total | 626 | 19.0 | 3.97 |
| Difficulty in falling asleep | 73 | 2.2 | 1.67 |
| Difficulty in staying asleep | 83 | 2.5 | 1.57 |
| Problem of waking too early | 81 | 2.5 | 1.26 |
| Sleep quality | 237 | 7.2 | 1.97 |
| Satisfaction with sleep | 104 | 3.2 | 0.32 |
| Sleep and daily life | 102 | 3.1 | 0.40 |
| Sleep and quality of life | 86 | 2.6 | 0.43 |
| Sleep and anxiety/depression | 97 | 2.9 | 0.18 |

TABLE 2

| | Total | Mean | Variance |
|---|---|---|---|
| Total | 626 | 19.0 | 3.97 |
| Sleep quality | 237 | 7.2 | 1.97 |

Generally speaking, the patients included mainly experience difficulties in staying asleep (mean score of 2.5) and problems of waking too early (mean score of 2.5). The value of these scores corresponds to a perception of the problem according to moderate to severe intensity. Satisfaction with sleep is assessed as distinctly inadequate by the patients who notice the major interference of the sleep problems in their daily life and in their quality of life. The poor sleep quality markedly “worries and depresses” these patients.

### 3.2) Intermediate Visit Analysis (D30)

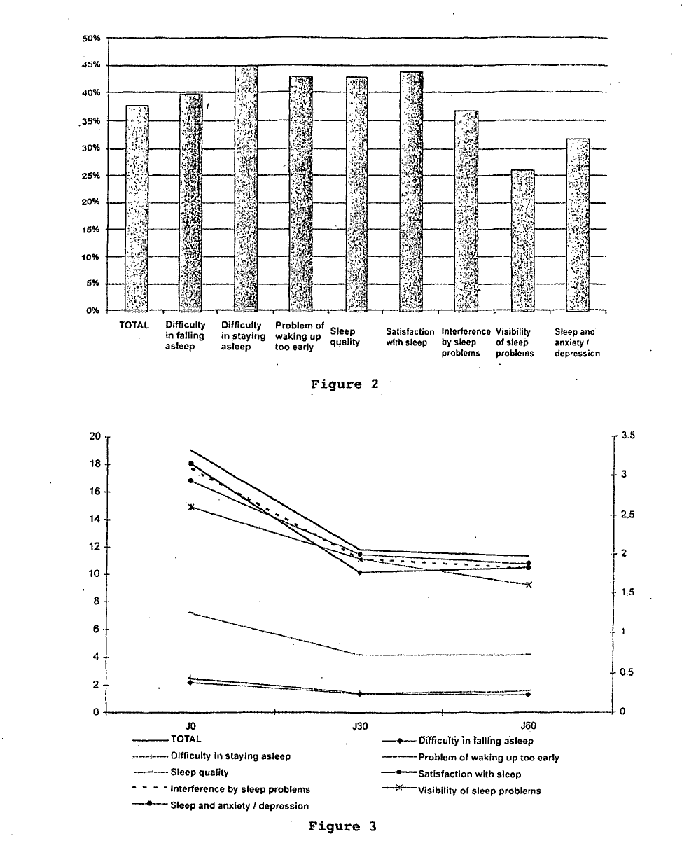

Tables 3 and 4 below show a major improvement in all the items in the patients after one month of administration of the product HB1 (refer to FIG. 2). The mean evolution over the total scores of all the items is −38%, i.e., the overall mean has improved and progresses from a value of 19 at D0 to a value of 11.8 at D30. This evolution places the overall score of the group of patients below the limit value on inclusion (15). It clearly shows the improvement in sleep provided by the product HB1. More specifically, one notes differences in improvement among the different criteria. The criterion with the most favorable evolution is “difficulty in staying asleep,” a criterion which indicates the frequency and intensity of insomnia.

TABLE 3

| | | | Varia- | Vari- | Student's |
|---|---|---|---|---|---|
| | Total | Mean | tion/DO | ance | test* |
| Total | 388 | 11.8 | −38% | 17.19 | <0.01 |
| Difficulty in falling | 44 | 1.3 | −40% | 0.92 | <0.01 |
| asleep | | | | | |
| Difficulty in staying | 46 | 1.4 | −45% | 1.06 | <0.01 |
| asleep | | | | | |
| Problem of waking | 46 | 1.4 | −43% | 0.81 | <0.01 |
| too early | | | | | |
| Satisfaction with sleep | 58 | 1.8 | −44% | 1.13 | <0.01 |
| Sleep and daily life | 64 | 1.9 | −37% | 0.87 | <0.01 |
| Sleep and quality of life | 64 | 1.9 | −26% | 0.62 | <0.01 |
| Sleep and anxiety . | 66 | 2.0 | −32% | 0.56 | <0.01 |
| depression | | | | | |

*Student's test: period n/period n-1

TABLE 4

| | | | Varia- | Vari- | Student's |
|---|---|---|---|---|---|
| | Total | Mean | tion/DO | ance | test* |
| Total | 388 | 11.8 | −38% | 17.19 | <0.01 |
| Sleep quality | 136 | 4.1 | −43% | 3.55 | <0.01 |

*Student's test: period n/period n-1

The other criteria are all less than or equal to 2, which implies scoring at a level considered low or moderate in relation to the different situations. As expected, all these variations are statistically significant (Student's test).

### 3.3) End of Study Visit Analysis (D60)

Tables 5 and 6 below show a marked improvement at D60 in relation to D0 which confirms the values obtained at D30. This improvement shows a positive tendency in relation to D30, without the values being statistically significant, except for the criterion “interference of sleep problems in quality of life” (FIG. 3).

TABLE 5

| | | | Varia- | Vari- | Student's |
|---|---|---|---|---|---|
| | Total | Mean | tion/DO | ance | test* |
| Total | 371 | 11.2 | −4% | 24.81 | NS |
| Difficulty in falling | 42 | 1.3 | −5% | 0.95 | NS |
| asleep | | | | | |
| Difficulty in staying | 43 | 1.3 | −7% | 1.09 | NS |
| asleep | | | | | |
| Problem of waking | 51 | 1.5 | 11% | 1.01 | NS |
| too early | | | | | |
| Sleep quality | 136 | 4.1 | 0% | 3.55 | NS |
| Satisfaction with sleep | 60 | 1.8 | 3% | 1.34 | NS |
| Sleep and daily life | 60 | 1.8 | −6% | 0.90 | NS |
| Sleep and quality of life | 53 | 1.6 | −17% | 0.81 | <0.01 |
| Sleep and anxiety/ | 62 | 1.9 | −6% | 0.98 | NS |
| depression | | | | | |

*Student's test: period n/period n-1

TABLE 6

| | | | Varia- | Vari- | Student's |
|---|---|---|---|---|---|
| | Total | Mean | tion/DO | ance | test* |
| Total | 371 | 11.2 | −4% | 24.81 | NS |
| Sleep quality | 136 | 4.1 | 0% | 3.55 | NS |

*Student's test: period n/period n-1

Interpretation of the data shows that the second month of treatment makes it possible to confirm the positive effects obtained during the first month. The efficacy of HB1 is consolidated and maintained in this case.

1-12. (canceled)

13. A composition containing polyunsaturated fatty acids and flavonoids as active substances.

14. The composition according to claim 13, wherein the polyunsaturated fatty acids are alpha-linolenic acid (C103n-3) and linoleic acid (C18:2n-6).

15. The composition according to claim 14, wherein the alpha-linolenic and linoleic acids are vegetable oils rich in polyunsaturated fatty acids.

16. The composition according to claim 15, wherein the vegetable oils are hemp oil or soybean oil.

17. The composition according to claim 13, wherein the flavonoids are present in the form of an extract of Humulus lupulus.

18. The composition according to claim 13, wherein the quantity of extract and oil in the composition is such that it allows a daily supply of:
Humulus lupulus (dry extract of hop cone powder): about 20 to about 1000 mg;
vegetable oil(s) rich in polyunsaturated fatty acids: about 400 to about 3000 mg, corresponding to:
about 300 to about 2600 mg of linoleic acid,
about 60 to about 600 mg of alpha-linolenic acid.

19. The composition according to claim 13, comprising:
about 10 to about 500 mg of extract of Humulus lupulus, and
about 200 to about 1500 mg of vegetable oil(s) rich in polyunsaturated fatty acids, corresponding to:
about 150 to about 1300 mg of linoleic acid,
about 30 to about 300 mg of alpha-linolenic acid.

20. A method of treating disorders caused by disturbance of a biological clock in humans and animals comprising administering a therapeutically effective amount of the composition of claim 1 to a human or animal.

21. The method according to claim 20, wherein the disorders caused by disturbance of the biological clock are selected from the group consisting of dyssomnia, parasomnia and sleep disturbances of psychiatric or neurological origin.

22. The method according to claim 20, wherein the disturbances are selected from the group consisting of difficulty in falling asleep, poor sleep quality, difficulty in waking up, major fatigue during the daytime and consequently memory disturbances, difficulties in concentration and orientation in space.

23. The method according to claim 20, wherein the composition contains a quantity of extract of Humulus lupulus and one or several vegetable oil(s) rich in polyunsaturated fatty acids capable of allowing a daily supply of:
Humulus lupulus: about 20 to about 1000 mg;
vegetable oil(s) rich in polyunsaturated fatty acids: about 400 to about 3000 mg, corresponding to:
about 300 to about 2600 mg of linoleic acid,
about 60 to about 600 mg of alpha-linolenic acid.

24. The method according to claim 20, wherein the polyunsaturated fatty acids are alpha-linolenic acid (C103n-3) and linoleic acid (C18:2n-6).

25. The composition according to claim 24, wherein the alpha-linolenic and linoleic acids are vegetable oils rich in polyunsaturated fatty acids.

26. The composition according to claim 25, wherein the vegetable oils are hemp oil or soybean oil.

27. The composition according to claim 20, wherein the flavonoids are present in the form of an extract of Humulus lupulus.

28. A method of regulating the biological clock in a mammal comprising administering a therapeutically effective amount of the composition of claim 13 to the mammal.

29. The method according to claim 28, wherein the composition contains a quantity of extract of Humulus lupulus and one or several vegetable oil(s) rich in polyunsaturated fatty acids capable of allowing a daily supply of:
Humulus lupulus: about 20 to about 1000 mg;
vegetable oil(s) rich in polyunsaturated fatty acids: about 400 to about 3000 mg, corresponding to:
about 300 to about 2600 mg of linoleic acid,
about 60 to about 600 mg of alpha-linolenic acid.

30. The method according to claim 29, wherein the polyunsaturated fatty acids are alpha-linolenic acid (C103n-3) and linoleic acid (C18:2n-6).

31. The composition according to claim 30, wherein the alpha-linolenic and linoleic acids are in the form of vegetable oils rich in polyunsaturated fatty acids.

32. The composition according to claim 31, wherein the vegetable oils are hemp oil or soybean oil.

33. The composition according to claim 29, wherein the flavonoids are present in the form of an extract of Humulus lupulus.
